(19) United States
(12) Patent Application Publication
(10) Pub. No.: US 2005/0282270 A1
(43) Pub. Date: Dec. 22, 2005

(54) System for thermally cycling biological samples with heated lid and pneumatic actuator

(75) Inventors: Hon Siu Shin, Singapore (SG); Jew Kwee Ngui, Singapore (SG); Yang Hooi Kee, Johor (MY); Lim Hi Tan, Singapore (SG)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/873,741

(22) Filed: Jun. 21, 2004

(51) Int. Cl. C12M 1/38

(52) U.S. Cl. 435/303.1; 435/305.3; 435/286.1; 435/91.2

(57) ABSTRACT

Systems and methods for thermal cycling samples that include pneumatic automation are provided by the present teachings.

### FIELD

The present teachings generally relate to thermal cycling biological samples particularly to systems for thermal cycling including a heated lid and a pneumatic actuator.

Introduction

Thermal cycling can be used to amplify nucleic acids by, for example, performing polymerase chain reactions (PCR) and other reactions for endpoint or real-time analysis. Thermal cycling devices require insertion of biological samples. It is desirable to provide devices and methods for performing thermal cycling that include automation in loading the thermal cycling device.

### SUMMARY

In various embodiments, a system for thermal cycling samples is provided. In various embodiments, the system comprises at least one sample well tray with a plurality of sample wells, at least one thermal cycling device having a plurality of cavities to receive at least a portion of the sample wells, at least one heated lid, and at least one pneumatic driver connected to the heated lid. In various embodiments, the pneumatic driver is configured to position the heated lid in a closed position and an open position, and to move the heated lid between the closed position and the open position. In various embodiments, the system further comprises at least one pneumatic actuator connected to the pneumatic driver, the pneumatic actuator configured to actuate the pneumatic driver to position and move the heated lid between the closed position and the open position. In various embodiments, the system further comprises a controller coupled to the pneumatic actuator, the controller configured to provide an electric signal to the pneumatic actuator to actuate the pneumatic driver.

In various embodiments, the present teachings can provide a method for thermal cycling samples including providing biological samples in a plurality of sample wells, positioning the sample wells in a thermal cycling device, pre-charging a pneumatic driver, closing a heated lid with the pneumatic driver, locking the heated lid with the pneumatic driver, thermally cycling the biological samples, unlocking the heated lid with the pneumatic driver, opening the heated lid with the pneumatic driver, and removing the sample wells from the thermal cycling device.

It is to be understood that both the foregoing general description and the following detailed description of various embodiments are exemplary and explanatory only and are not restrictive. This and other features of the present teachings will become more apparent from the description herein.

### BRIEF DESCRIPTION OF THE DRAWINGS

The accompanying drawings, which are incorporated in and constitute a part of this specification, illustrate various exemplary embodiments. The skilled artisan will understand that the drawings, described below, are for illustration purposes only. The drawings are not intended to limit the scope of the present teachings in any way.

FIG. 1 is a front perspective view of an exemplary embodiment of a system for thermal cycling biological samples according to the present teachings, with the lid in a closed position;

FIG. 2 is a rear perspective view of the system of FIG. 1, with the lid in the closed position;

FIG. 3 is a front view of the system of FIG. 1, with a portion of a housing removed for illustration purposes;

FIG. 4 is a front perspective view of the system of FIG. 1, with a handle of the lid rotated into an upward position;

FIGS. 5A-5B is a front perspective view of the system of FIG. 1, with the lid in an open position, FIG. 5A showing the multi-well tray loaded in the thermal cycling device and FIG. 5B showing the recesses in the block;

FIG. 6 is a circuit diagram of the pneumatic actuator and pneumatic cylinders, according to the present teachings; and

FIG. 7 is a flow chart of the control logic for the pneumatic actuator of FIG. 6, according to the present teachings;

FIG. 8 is a diagram of a system with a plurality of thermal cycling devices and a plurality of controllers, according to the present teachings; and

FIG. 9 is a diagram of a system with a plurality of thermal cycling devices and a single controller, according to the present teachings.

### DESCRIPTION OF VARIOUS EMBODIMENTS

Reference will now be made to various exemplary embodiments, examples of which are illustrated in the accompanying drawings. Wherever possible, the same reference numbers are used in the drawings and description to refer to the same or like parts. Although terms such as “horizontal,” “vertical,” “upward,” and “downward” are used in describing various aspects of the present teachings, it should be understood that such terms are for purposes of more easily describing the teachings, and do not limit the scope of the teachings. The complete disclosures of all publications discussed herein are hereby incorporated by reference for any purpose. In the event that one or more of the incorporated literature and similar materials differs from or contradicts this application, including but not limited to defined terms, term usage, described techniques, or the like, this application controls.

In various embodiments, a system for thermal cycling biological samples can include a thermal cycling device, a heated lid, recesses for at least one sample well tray with a plurality of sample wells, and at least one pneumatic driver connected to the heated lid to position the heated lid in a closed position and an open position. In various embodiments, the system can further include one or more of: a pneumatic actuator connected to the pneumatic driver, the pneumatic actuator configured to actuate the pneumatic driver to position and move the heated lid between the closed position and the open position; and a controller coupled to the pneumatic actuator, the controller configured to provide an electric signal to the pneumatic actuator.

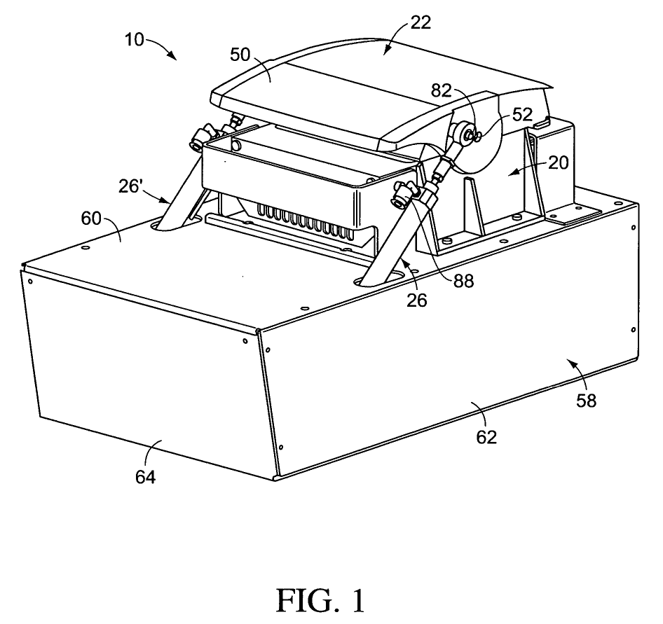

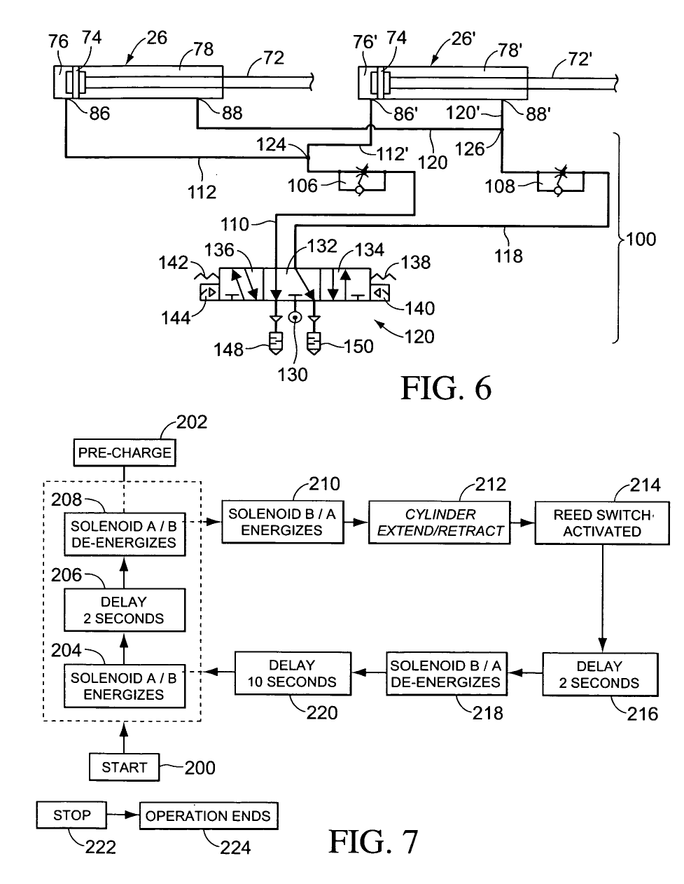

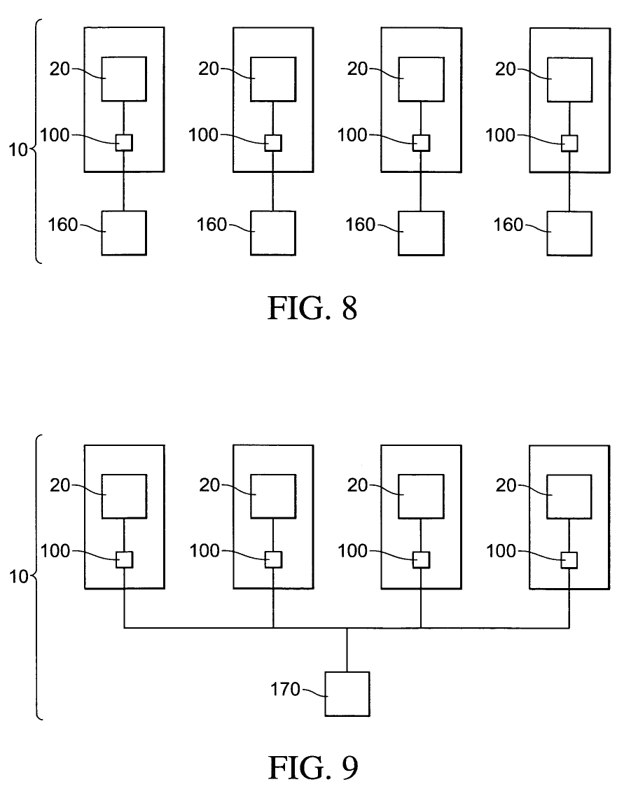

Various aspects of the present teachings can be further understood in light of the following examples, which should not be construed as limiting the scope of the present teachings in any way. In various embodiments illustrated in FIGS. 1-5, the system 10 for thermally cycling biological samples can include a thermal cycling device 20, a heated lid 22, recesses for at least one sample well tray 24 with a plurality of sample wells, and at least one pneumatic driver 26 connected to the heated lid 22. The system can also include a pneumatic actuator 100 (see FIGS. 6 and 8) and a controller.

The system for thermal cycling biological samples includes a thermal cycling device. Various embodiments of a thermal cycling device are shown in FIGS. 1-5. As illustrated in FIGS. 1-5, the thermal cycling device is designated by reference number 20. In various embodiments, the thermal cycling device can be configured to perform nucleic acid amplification on samples. One common method of performing nucleic acid amplification of samples is polymerase chain reaction (PCR). Various exemplary PCR methods are known in the art, as described in, for example, U.S. Pat. Nos. 5,928,907 and 6,015,674 to Woudenberg et al. Various other exemplary methods of nucleic acid amplification include, but are not limited to, ligase chain reaction, oligonucleotide ligation assay, and hybridization assay. Various examples of these and other methods are described in greater detail in U.S. Pat. Nos. 5,928,907 and 6,015,674.

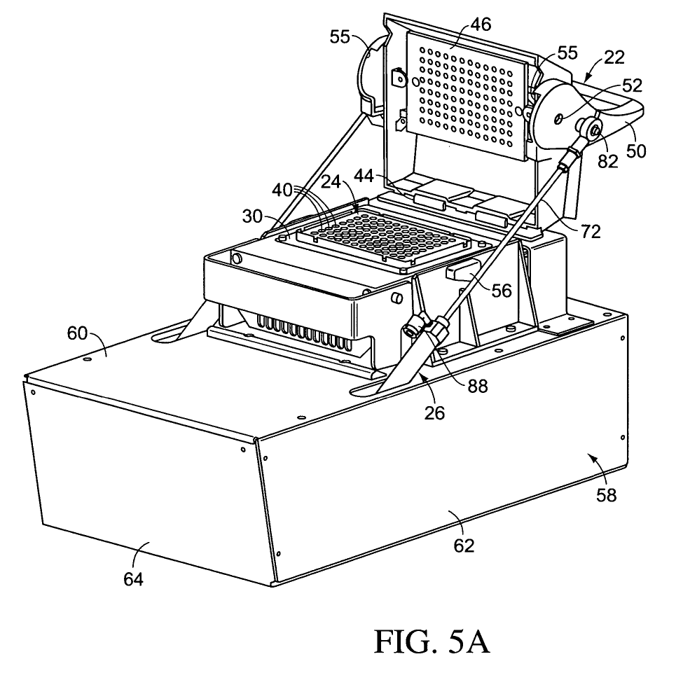

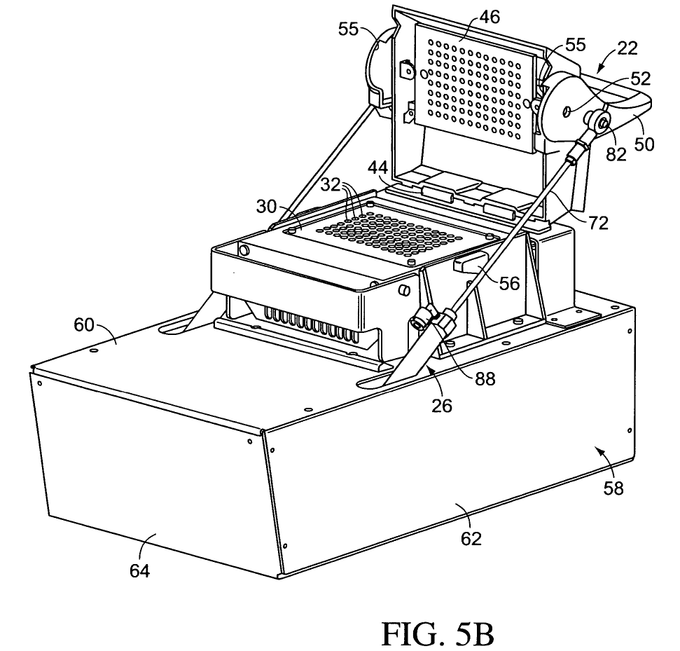

The thermal cycling device can be of any type that is suitable for performing thermal cycling. As illustrated in FIG. 5, the thermal cycling device 20 can include a sample block 30 with a plurality of cavities or recesses shown in FIG. 5B for receiving a portion of sample wells of the sample well trays therein. In various embodiments, the sample block provides a plurality of cavities in a top portion thereof for receiving a bottom portion of the sample well tray. The recesses can have any suitable shape, such as a conical shape, which is sized to fit with a sample well of the sample well tray. In various embodiments, the sample block cavities can be other shapes such as cylindrical or hemispherical, depending on the shape of the mating sample wells. In various embodiments, the sample block can be flat without recesses such that is can couple to micro-card, such as 384-well microcard, or a tray where the wells do not project individually from the bottom of the tray, such as a 1536-well sample tray or a 6144-well sample tray.

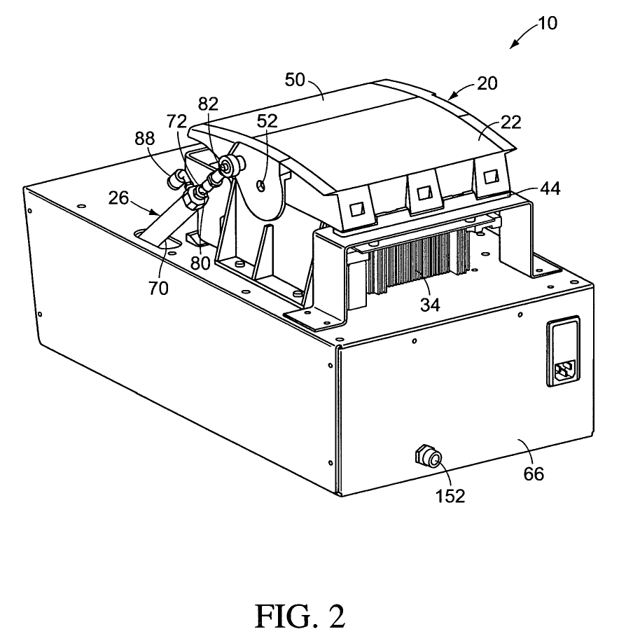

In various embodiments, the sample block can be made out of any suitable material that can be raised and lowered to suitable temperatures for thermal cycling. In various examples, the sample block is a metal such as aluminum or aluminum alloy or any thermally conductive material, including thermally conductive composites and plastics. In various embodiments, the sample block can be attached to any other suitable heating and cooling structures, such as cooling fins 34 shown in FIG. 2. Various other heating and cooling structures such as thermoelectric coolers, resistive heaters, and fans can also be provided in various embodiments.

In various embodiments, the thermal cycling device, including the sample block, can be configured for receiving any suitable type of sample well tray. In the example shown in FIGS. 5A-5B, the thermal cycling device 20 is configured to receive one 96-well sample tray 24. The sample well tray 24 includes ninety-six sample wells 40 positioned in an 8×12 matrix on the tray. It is understood that the present teachings are also suitable with other configurations, such as, but not limited to, a dual 96-well tray configuration, single or dual 384-well tray configurations, and other single or dual configurations such as 60-well, 1536-well, or 6144-well configurations. Other configurations with any number of sample wells ranging from one sample well to several thousand (e.g. 4, 16, 24, 48, 96, 384, 1536, 6144, etc.) can also be utilized in various embodiments. In various embodiments, the sample wells are configured for containing a predefined volume of liquid sample. Although the figures illustrate the sample wells being integrally formed as part of the sample well tray, in various embodiments, individual tubes or tube strips can be sample holders. In various embodiments, the tubes can be connected together in sets of rows or columns. As can be readily understood, any type of suitable sample well tray can be used within various embodiments.

In various embodiments, the system for thermally cycling samples further includes a heated lid. As shown in various embodiments in FIGS. 1-5, the heated lid is designated by reference number 22. In various embodiments, the heated lid 22 is movable between various positions. In the example shown in FIGS. 1-5, the heated lid 22 is pivotable about the thermal cycling device 20 between a closed position shown in FIGS. 1-3, and an open position shown in FIGS. 5A-5B. In the example shown in FIGS. 1-5, the heated lid includes a pivoting hinge 44 for permitting pivoting of the heated lid about the axis of pivoting hinge 44. In various embodiments, the pivoting hinge 44 can be of any suitable type. In various embodiments, the heated lid can be linearly translatable as described in WO 00/146688 A1, or stationary and the block movable as described in U.S. Pat. No. 6,677,151.

In various embodiments, the heated lid can be of any suitable type. The heated lid can be of the type that permits real time detection of the samples during thermal cycling. In various embodiments, the heated lid can be configured for endpoint detection of the samples after thermal cycling is performed. In various embodiments, the heated lid can further include a heated platen, such as heated platen 46 shown in FIGS. 5A-5B. In a various embodiments, the heated platen is configured for pressing down on the top surface of the sample well tray, or on caps on the top of each sample well. In various configurations, the heated platen can assist in preventing or minimizing condensation on the top portion of the sample wells of the sample well tray, when the heated lid is in its closed position. In various embodiments, the top portion of the sample wells of the sample well tray is defined by a cap, adhesive film, heat seal, and/or gap pad (not shown). In various embodiments, the heated lid can be closed so that the heated portion engages the top portion of the sample wells by pivoting from the open position shown in FIGS. 5A-5B to a downward or closed position shown in FIGS. 1-3, as will be described in greater detail when discussing the pneumatic actuator according to various embodiments.

In the example shown in FIGS. 1-5, the heated lid also includes a handle that is pivotably connected to the main body portion of the heated lid. In the example shown in FIGS. 1-5, handle 50 is rotatable about a rotational axis at pivot 52 on the heated lid 22. In various embodiments, during normal operation, the handle is pivoted between a downward and an upward position by the pneumatic apparatus. In various embodiments, the handle can also be used as a fail safe manual method for opening and closing the heated lid, upon a failure of the pneumatic apparatus. In such a situation, a user could manually grab forward portion 54 of handle 50 with his or her hand to open or close the handle and movable lid. The pivoting action of the handle of the heated lid during normal operation in various embodiments will be discussed in greater detail when discussing the pneumatic driver.

In various embodiments, the system can further include a locking device configured to lock the heated lid onto the thermal cycling device when the heated lid is in a closed position. In the example shown in FIGS. 5A-5B, the locking device includes an opening 55 in the handle for receiving a cam structure 56 attached to a side wall of the sample block 30. As the heated lid 22 is pivoted to the closed position, the cam structure 56 can be received within the opening 55 of the handle 50 so that it engages with a corresponding cam or locking structure of the handle as the handle is rotated about the pivot 52 to its downward position. In various embodiments, as the handle is moved to its fully downward position, the locking device can provide an additional downward force on the heated lid so that it is securely positioned on the thermal cycling device. In various embodiments, the locking device can assist in ensuring that the heated lid is not opened when the handle 50 is in the downward position, such as when thermal cycling is being performed on the sample well tray. In various embodiments, the locking device can be any suitable locking structure that is capable of preventing opening of the heated lid when the handle is rotated to the downward position. Although the locking device shown in FIGS. 1-5 is a mechanical structure that is engaged as the handle is rotated about the heated lid, other types of locking devices such as an electrically actuated device, or a pneumatically actuated device can also be used in various embodiments. For example, instead of the cam structure 56, in various embodiments, any suitable type of electronically actuated locking system can be utilized.

In various embodiments, the thermal cycling device and heated lid can be mounted within the system in any suitable manner. In the example shown in FIGS. 1-5, the thermal cycling device is mounted on a housing 58. In FIGS. 1-5, housing 58 is generally rectangular with a top surface 60, side walls 62, a front wall 64, and a rear wall 66. The housing is shown having a generally rectangular shape, however, any other suitable shape is also acceptable, in various embodiments. Moreover, in various embodiments, the housing can be used to contain any number of devices and accessories of the system.

In various embodiments, the system for thermally cycling biological samples further includes at least one pneumatic driver connected to the heated lid, the pneumatic driver configured to position the heated lid in a closed position and an open position, and to move the heated lid between the closed position and the open position. In various embodiments, the system further includes at least one pneumatic actuator connected to the pneumatic driver, and a controller coupled to the pneumatic actuator. In various embodiments shown in FIGS. 1-6, the pneumatic driver includes a pair of reciprocating pneumatic cylinders 26 and 26′. In the embodiments shown in FIGS. 1-8, the pair of pneumatic cylinders are symmetrically positioned about the thermal cycling device. It should be understood that in various embodiments the two pneumatic cylinders are identical, therefore, the structure of only one of the pneumatic cylinders will typically be described below. In various embodiments, one, two, or more pneumatic cylinders can be used.

In various embodiments, the pneumatic cylinders are rotatably mounted to a stationary member at a first end thereof. In the example shown in FIGS. 1-5, the pneumatic cylinder is pivotably connected via pin 68 to a stationary object such as the housing 58 of the system, or to a fixed horizontal surface. In the example shown in FIGS. 1-5, each pneumatic cylinder 26 includes an outer cylinder 70 and an inner rod 72. Inner rod 72 is configured to translate within outer cylinder 70. The lower end of inner rod 72 includes a piston 74 that is slidingly and sealingly engaged within the outer cylinder 70. Piston 74 defines two chambers within the outer cylinder—lower chamber 76 and upper chamber 78, as illustrated in FIG. 6. By varying the pressure (and amount of air) in each of the chambers, the piston 74 will move linearly within the outer cylinder 70. Rod 72 extends from the outer cylinder 70 through a sealed fit with the outer cylinder 70 at hole 80 (see FIG. 2). On the upper end of the rod 72 opposite the piston 74 is a pin 82. In the example shown, pin 82 is rotatably connected to the handle 50 of the heated lid 22.

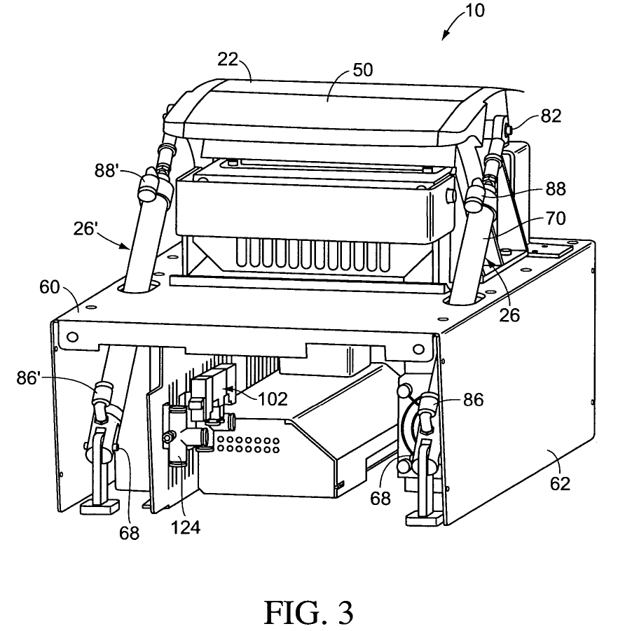

In various embodiments, the pneumatic driver can include air ports 86 and 88 for permitting air to enter and exit each of the chambers 76 and 78 of the outer cylinder 70. In the example shown in FIGS. 1-5, elbow joints are provided for the air ports of the chambers. More particularly, as shown in FIG. 3, for example, a lower elbow joint for air port 86 can be provided at a lower end of the outer cylinder 70 adjacent pin 68 and an upper elbow joint for air port 88 can be provided at an upper end of the outer cylinder 70. The elbow joints permit air to enter and exit the respective chambers to cause the piston 74 to translate within the outer cylinder 70. For example, if the force created by the air in lower chamber 76 is greater than the counter force caused by the air in upper chamber 78 (and any other downward forces), the piston 74 will translate in a generally “upward” direction to extend the rod 72 from the outer cylinder 70. This upward motion of the pneumatic cylinder is referred to as “extension” of the cylinder. Conversely, if the force created by the air in upper chamber 78 is greater than the counterforce caused by the air in lower chamber 76, the piston 74 will translate in a generally downward direction to retract the rod 72 into the outer cylinder 70. This downward motion of the pneumatic cylinder is referred to as “retraction” of the cylinder.

An example of the general positions and movements of the pneumatic cylinder and heated lid for various embodiments will first be illustrated. In various embodiments, the pneumatic cylinder has three basic positions: a first, second, and third position. The first position for the pneumatic cylinder is shown in FIGS. 1-3. In FIGS. 1-3, the rod 72 of the pneumatic cylinder is retracted within the outer cylinder, and the heated lid 22 and the handle 52 are in their lowest downward positions. For purposes of this description, this position shown in FIGS. 1-3 is referred to as the closed position of the heated lid, and the downward position of the handle. In the closed position, the heated lid is closed over the sample well tray, typically pressing downward on the top surface of the sample well tray in a manner described above. It is in this first position that thermal cycling is performed by the thermal cycling device. In this first position, lower chamber 76 has its smallest volume of air, and upper chamber 78 has its largest volume of air. The pneumatic cylinder is moved to this position by air being forced into the upper chamber 78 via upper air port 88, and air being allowed to exit lower chamber 76 via lower air port 86.

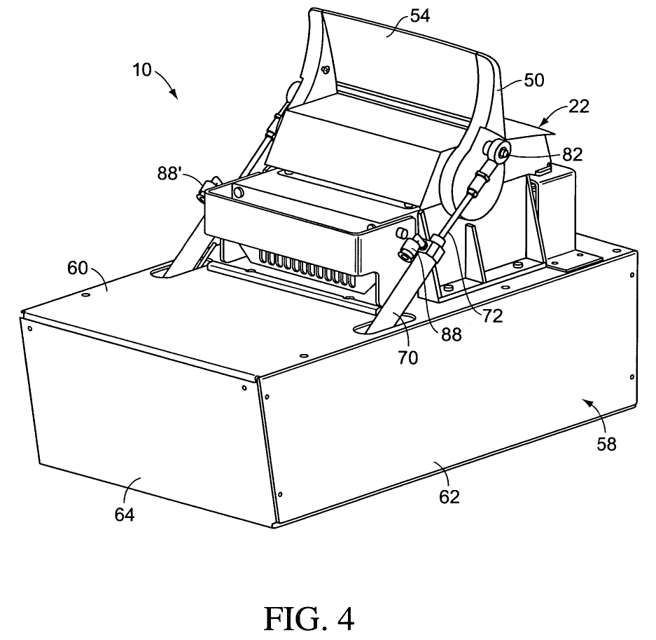

The second position for the pneumatic cylinder is shown in FIG. 4. In FIG. 4, the rod 72 of the pneumatic cylinder has extended out of the outer cylinder relative to the first position shown in FIGS. 1-3 and described above. The handle 50 has also been caused to rotate about the axis of pivot 52 of the heated lid 22 from the downward position shown in FIGS. 1-3 to its upward position (shown in FIG. 4). The heated lid 22 however remains in the closed position described in FIGS. 1-3. This second position for the pneumatic cylinder, shown in FIG. 4, is also referred to as the “intermediate” position for the pneumatic cylinder. It is at this position that any additional extension of the pneumatic cylinder will cause the heated lid to begin to open. In the intermediate position shown in FIG. 4, the locking device of the handle is no longer engaged, so that the locking device does not prevent the heated lid from being pivoted about its axis to open the heated lid.

Upon further extension of the pneumatic cylinder from the position shown in FIG. 4, the pneumatic cylinder reaches its third position, shown in FIGS. 5A-5B. In FIGS. 5A-5B, the heated lid is in its fully open position and the handle portion remains in its upward position. In this position, the sample well tray (or trays) are accessible for removal from the thermal cycling device as illustrated in FIG. 5A or lading as illustrated in FIG. 5B. In various embodiments, the sample well tray can be removable automatically, such as by a robot, or removable manually. In this third position for the pneumatic cylinder, shown in FIGS. 5A-5B, air has been allowed to enter lower air port 86 to fill lower chamber 76, and air has exited upper chamber 78 via upper air port 88. It should be understood that, in addition to the three main positions for the pneumatic cylinder discussed above, in various embodiments, there are an infinite number of positions therebetween.

In various embodiments, the system for thermal cycling samples further includes at least one pneumatic actuator connected to the pneumatic driver, and a controller coupled to the pneumatic actuator. In various embodiments, the pneumatic actuator is configured to actuate the pneumatic cylinders to position and move the heated lid between the closed position and the open position. In various embodiments, as illustrated in FIG. 6, the pneumatic actuator is generally designated by reference number 100. In various embodiments, the pneumatic actuator is used to selectively provide pneumatic pressure to the lower and upper chambers of the pneumatic cylinders 26, to cause the pin 72 to extend and retract, thereby opening and closing the heated lid 22 of the thermal cycling device. In various embodiments, the pneumatic actuator includes one or more of a multi-position solenoid valve and a flow controller.

In various embodiments, as illustrated in the block diagram of FIG. 6, a solenoid valve 102 can be provided for selectively providing pneumatic flow to a pair of flow controllers 106 and 108. In the example shown in FIG. 6, a pneumatic line 110 runs from the solenoid valve 102 to the first flow controller 106. The pneumatic line 110 then continues and branches into pneumatic lines 112 and 112′ at T-joint 124. Although the pneumatic lines are not shown in FIG. 3 for ease of description, FIG. 3 shows an example of a T-joint 124 that can be used for branching pneumatic line 110 into pneumatic lines 112 and 112′. In various embodiments, any other suitable type of joint can also be used. As shown in FIG. 6, pneumatic line 112 leads to lower air port 86 of pneumatic cylinder 26, and pneumatic line 112′ leads to lower air port 86′ of pneumatic cylinder 26′. In various embodiments, the lines are configured so that a substantially identical flow of air is supplied to pneumatic lines 112 and 112′ so that pneumatic cylinders 26 and 26′ extend and retract in a symmetrical manner. Pneumatic lines 110, 112, and 112′ are used to communicate with the lower chambers 76 and 76′ of the pneumatic cylinders 26 and 26′.

Similarly, a pneumatic line 118 runs from the solenoid valve 102 to the second flow controller 108. The pneumatic line 118 then continues and branches into pneumatic lines 120 and 120′ at T-joint 126. Although the pneumatic lines are not shown in FIG. 3 for ease of description, FIG. 3 shows an example of a T-joint 126 that can be used for branching pneumatic line 118 into pneumatic lines 120 and 120′. Pneumatic line 120 leads to upper air port 88 of pneumatic cylinder 26, and pneumatic line 120′ leads to upper air port 88′ of pneumatic cylinder 26′. In various embodiments, the lines are configured so that an identical flow of air is supplied to pneumatic lines 120 and 120′ so that pneumatic cylinders 26 and 26′ extend and retract in a symmetrical manner. Pneumatic lines 118, 120, and 120′ are used to communicate with the upper chambers 78 and 78′ of the pneumatic cylinders 26 and 26′. In various embodiments, the pneumatic lines described above can be any type of suitable tubing or piping. In various embodiments, the pneumatic lines are made out of polyurethane tubing or tubing made of other materials such as polyethylene, polyamide, polyvinyl chloride, etc. In various embodiments, other suitable materials can be used.

The flow controllers 106 and 108 are used in order to control the flow of air to and from the pneumatic cylinders. The flow controllers can regulate the flow to control the amount of air flowing to the cylinder chamber.

In various embodiments, the pneumatic actuator includes a solenoid valve. In the example shown in FIG. 6, the solenoid valve 102 provides pneumatic (air) pressure selectively to the pneumatic cylinder to cause the cylinders to extend or retract, or to remain in a fixed position. In the example shown in FIG. 6, the solenoid valve is a 5 port/3 position solenoid valve. In various embodiments, any other suitable valve can also be used. The specific example of the solenoid valve shown in FIG. 6 will be described below.

As embodied in FIG. 6, solenoid valve 102 includes an air inlet 130. Air inlet 130 of the solenoid valve can be connected to any suitable source of air pressure. In various embodiments, the source of air can be pressurized air that is pumped or channeled from a pressurized container or line. In various embodiments, the air source can be generated from an air compressor. As used herein, the term “air” refers to any pressurized gas such as nitrogen or oxygen that can be readily substituted. In the example shown in FIG. 6, air inlet 130 of the solenoid communicates with air inlet/outlet 152 (shown in FIG. 2) on the rear wall 66 of the housing 58, in any suitable manner, such as via tubing. The solenoid valve 102 shown in FIG. 6 further includes a fixed section 132, and first and second movable sections 134 and 136 on opposite sides of the fixed section 132. First movable section 134 includes a return spring 138 and a solenoid 140 (also referred to as “solenoid A”). Second moveable section 136 includes a return spring 142 and a solenoid 144 (also referred to as “solenoid B”). The movable sections 134 and 136 of the solenoid valve are selectively movable by their respective solenoids 140 (solenoid A) and 144 (solenoid B). The return springs are set to a biased position to substantially prevent flow into the pneumatic lines.

The solenoid valve 102 shown in FIG. 6 has three main positions: (1) a first position in which no air can flow into either of the pneumatic lines 110 or 118; (2) a second position in which air can flow into pneumatic line 110 to cause rods 72 and 72′ to extend; and (3) a third position in which air can flow into pneumatic line 118 to cause the rods 72 and 72′ to retract. In various embodiments, additional positions for the solenoids provide a fixed intermediate position for the heated lid. In various embodiments, the solenoid valve is generally configured to be in the first position, absent the energization of one of the solenoids. To obtain the second position of the solenoid valve, solenoid 144 (solenoid B) is energized, causing second movable section 136 to move from its original position toward the fixed section 132 of the solenoid valve, causing air to flow into the pneumatic line 110 and into the lower chambers 76 and 76′ of the pneumatic cylinders, thereby causing the inner rods 72 and 72′ to extend. When solenoid 144 is de-energized, second movable section 136 moves back to original position, due largely to the biasing force of return spring 142. To obtain the third position of the solenoid valve, solenoid 140 (solenoid A) is energized, causing first movable section 134 to move from its original position toward the fixed section 132 of the solenoid valve, causing air to flow into pneumatic line 118 and into the upper chambers 78 and 78′ of the pneumatic cylinders, thereby causing the inner rods 72 and 72′ to retract. When solenoid 140 is de-energized, first movable section 134 moves back to its original position, due largely to the biasing force of return spring 138. In the first solenoid valve position, no air can enter either of the pneumatic lines 110 or 118. Therefore, when all of the solenoids are de-energized, there should be substantially no movement by the pneumatic cylinders. Although FIG. 6 shows one type of suitable valve, a 5 port/3 position solenoid valve, in various embodiments, any other suitable type of valve can also be utilized.

In the embodiments shown in FIG. 6, silencers 148 and 150 can also be provided as shown. The function of the silencer can be to reduce the noise generated by the exhaust flow from the pneumatic line.

As described above, in various embodiments, the pneumatic actuator, including the solenoid valve and flow controllers, selectively supplies pneumatic pressure to the pneumatic cylinders 26 and 26′ to cause the cylinders to retract and extend. In various embodiments, the pneumatic actuator can include various structures in order to regulate the distance that the pneumatic cylinders retract and extend. For example, in various embodiments, the pneumatic actuator can include one or more switches for sensing the position of the cylinders. In the example shown FIGS. 1-8, switches can be provided for sensing the position of the inner rod 72 relative to the outer cylinder 70. For example, reed switches can be provided at the top of the outer cylinder 70 and at the bottom of the outer cylinder 70 to sense when the inner rod is fully extended and retracted, respectively. In the embodiments shown in FIGS. 1-8, an upper reed switch (not shown) can be positioned generally adjacent the upper air port 88 on the inside of the outer cylinder, and a lower reed switch (not shown) can be positioned generally adjacent the lower air port 86 on the inside of the outer cylinder. In various embodiments, the upper reed switch can be configured to sense when the inner rod 72 has extended to a predetermined position, so that an appropriate signal can be sent to prevent any further extension beyond such a position. Similarly, in various embodiments, the lower reed switch can be configured to sense when the inner rod 72 has retracted to a predetermined position, so that an appropriate signal can be sent to prevent any further retraction beyond such a position. As discussed below, in various embodiments, the reed switches and system can alternately be positioned to allow only limited amount of movement after they are activated. In various embodiments, the reed switches are only provided on one of the pneumatic cylinders, although the reed switches could be provided on both of the pneumatic cylinders. The operation of the reed switches according to various embodiments is described in greater detail with respect to the control logic block diagram of FIG. 7.

In various embodiments, the system also includes a controller coupled to the pneumatic actuator, the controller configured to provide an electric signal and/or pneumatic signal to the pneumatic actuator. In various embodiments of a system with a pneumatic actuator that includes a solenoid valve with one or more solenoids, the controller can provide an electrical signal and/or pneumatic signal to selectively energize and de-energize the solenoids. Energization of a solenoid typically causes the solenoid to extend or retract. In various embodiments, the controller can be any suitable type. In various embodiments, the controller includes a control circuit. In various embodiments, the controller can include a computer and/or programmable logic controller (PLC) with one or more control circuits. PLCs can provide options for inputs and outputs, memory, and CPU power and can be user or factory programmed.

In various embodiments, each pneumatic actuator has a corresponding controller. In such a system, there can be one or several thermal cycling devices and pneumatic actuators. An example of a system with a plurality of thermal cycling devices and pneumatic actuators where each pneumatic actuator has an independent corresponding controller is shown in FIG. 8. FIG. 8 shows a system with a plurality of pneumatic actuators 100 each having their own controller 160. As shown in FIG. 8, each pneumatic actuator 100 is coupled to an independent controller 160. In various more specific embodiments, the pneumatic actuator 100 includes the above described solenoid valve 102 with solenoids A and B. As shown in the embodiment of FIG. 8, the pneumatic actuator 100 is connected to the pneumatic driver and heated lid of the thermal cycling device 20 to move the heated lid between the opened and closed position. As can be seen, there is no connection between the adjacent controllers in the example shown in FIG. 8, although they can be positioned closely together. It should be understood that the system according to various embodiments can include any number of thermal cycling devices from one to several hundred, and an equal number of corresponding controllers.

In various embodiments, the system includes a single controller. FIG. 9 illustrates an example of a system 10′ with a plurality of thermal cycling devices 20 and pneumatic actuators 100 that are all controlled by a single controller 170. In various embodiments, the controller 170 can include a control circuit or a control computer with one or more control circuits. In various embodiments with a single controller, the controller can be programmed to control all of the actuators simultaneously or at different times. In various embodiments, the use of a single controller to control a plurality of pneumatic actuators can be more cost effective than providing a large number of individual controllers, depending on the specific application requirements. In various embodiments, any type of suitable controller is acceptable.

In various embodiments, as illustrated in FIG. 7, control logic can be sued for the opening and closing of a heated lid using a pneumatic driver and pneumatic actuator. The block diagram at FIG. 7 illustrates a general control sequence starting from a first position where the heated lid is closed (FIGS. 1-3). The heated lid will be in this first position during thermal cycling and immediately thereafter. The control sequence for a system having a single thermal cycling device, pneumatic actuator, and pneumatic driver will be described according to various embodiments, although it should be understood that the identical control sequence can be performed for a system having multiple thermal cycling devices, actuators, and drivers. In various embodiments, first, as shown at step 200 in FIG. 7, a signal is sent to start the procedure for opening the heated lid, typically after the thermal cycling operations have been performed on the biological samples in the sample well tray. In various embodiments, this signal can be from a controller. In various embodiments, this signal can be from a manually actuated button such as a “Start Button.” After the start signal is sent, the next step in various embodiments can be a “pre-charge” step.

In the example shown in FIG. 7, the pre-charge step is generally designated by reference number 202 and shown within the dashed lines of FIG. 7. The pre-charging step can be utilized in various embodiments in order to minimize the vibration that would otherwise occur at the beginning of an extension or retraction of the pneumatic cylinder 26. For example, FIG. 6 shows a position for the pneumatic actuator and driver where the heated lid is in its fully closed position. In this position, also shown in FIGS. 1-3, the inner rods 72 and 72′ are retracted to a position close to the bottom of the pneumatic cylinders 26 and 26′. When it is desirable to begin to extend the inner rods 72 and 72′ by providing air to the lower chambers 76 and 76′, the upper chambers 78 and 78′ can be substantially devoid of any air or air pressure. Because there is little or no air in the upper chambers, in various instances, the piston can begin to translate or extend very quickly when air is directed into the lower chambers, causing undesirable vibrations in the pneumatic cylinder. The feature of pre-charging, in various embodiments, can assist in minimizing the vibrations that would otherwise occur at the beginning of an extension or retraction of the inner rod 72. During the step of pre-charging, the empty chamber of the cylinder (such as upper chamber 78 in the example discussed above) can be partially filled with air prior to any extension stroke in order to provide an initial cushioning of the piston when the extension stroke begins. From the position shown in FIG. 6, in various embodiments, pre-charging can occur by energizing solenoid A (reference number 140 in FIG. 6) so that air can flow into the pneumatic line 118 for a limited duration, such as 2 seconds, as shown by steps 204 and 206. In various embodiments, after the limited duration, solenoid A is de-energized (at reference number 208) in order to prevent further flow of air into pneumatic line 118. During the period in which the solenoid A is energized, an appropriate amount of air is allowed to flow into the upper chambers 78 and 78′ of the pneumatic cylinders. In various embodiments, the amount of air that is inserted into the upper chambers 78 and 78′ during pre-charging is sufficient to prevent a sudden jerking of the piston upward, but small enough to permit the lower chambers 76 and 76′ to be filled with air and move the piston upward in the next step (described below).

In various embodiments, after the pre-charging step 202 is completed and solenoid A is de-energized, the next step is to extend the pneumatic cylinders. In order to extend the pneumatic cylinders, solenoid B (reference number 144 in FIG. 6) is energized, thereby permitting flow through pneumatic line 110 to the lower chambers 76 and 76′ as previously described. As a result of this flow of air into the lower chambers 76 and 76′, the pneumatic cylinders extend as designated in step 212 of FIG. 7. As the pneumatic cylinders extend, the handle 50 of the heated lid 22 moves to the position shown in FIG. 4—the intermediate position. As the pneumatic cylinders continue to extend, the heated lid 22 then begins to open, pivoting about pivoting hinge 44 to approach the fully open position shown in FIGS. 5A-5B. In the embodiments shown in FIG. 7, the reed switch is placed at a predetermined position toward the top of the pneumatic cylinder. In the FIG. 7 embodiments, the reed switch at the top of the pneumatic cylinder is activated (or “turned on”) at step 214. In the embodiments shown in FIG. 7, there is a predetermined delay after the reed switch is activated. In various embodiments, the delay is approximately 2 seconds, as shown in step 216 of FIG. 7. After step 216, the controller sends a signal (or removes a signal) to the solenoid B (144) in order to de-energize solenoid B, as shown in step 218 of FIG. 7.

At the end of step 218, in various embodiments, a delay can be provided. In the example shown in FIG. 7, the delay can be for any appropriate period of time, such as 10 seconds, as shown in step 220 of FIG. 7. In various embodiments, the delay can be appreciably longer than 10 seconds. During the delay (step 220), the heated lid 22 is in the open position shown in FIGS. 5A-5B. In this open position shown in FIG. 5A, the sample well tray (or trays) 24 can be removed from the sample block 30 of the thermal cycling device 20. In various embodiments, the sample well tray can be removed by any suitable method. In various embodiments, the sample well tray is removed by a robotic device such as a robotic arm. In various embodiments, the sample well tray is removed manually, such as by an operator using his or her hands or by using a handheld removal tool. In various embodiments, after the sample well tray is removed from the surface of the sample block of the thermal cycling device, a new sample well tray can be inserted into the thermal cycling device, typically onto the sample block.

After the new sample well tray has been inserted into thermal cycling device, the heated lid can now be closed. The heated lid is now in the fully open position, with the pneumatic cylinders in their fully extended position. Prior to beginning to retract the pneumatic cylinders, in various embodiments, it can be desirable to provide the optional pre-charge step 202 in order to minimize vibrations and prevent sudden movement of the pneumatic cylinder in the downward direction. The pre-charge step prior to retraction of the pneumatic cylinder can be applied to the lower chambers 76 and 76′ of the pneumatic cylinder, instead of the upper chambers 78 and 78′ as described above, immediately prior to the extension of the pneumatic cylinders. In various embodiments, prior to retraction, pre-charging can occur by energizing solenoid B (reference number 144 in FIG. 6) so that air can flow into pneumatic line 110 for a limited duration, such as two seconds. In various embodiments, during the period in which solenoid B is energized (step 202), an appropriate amount of air is allowed to flow into the lower chambers 76 and 76′ of the pneumatic cylinders. In various embodiments, this amount of air is sufficient to prevent a sudden jerking of the piston downward (during step 210), but small enough to permit the upper chambers 78 and 78′ to be filled with air and move the piston downward in the next step (described below). In various embodiments, after the limited duration of the pre-charge, solenoid B is de-energized (at reference number 208) to prevent further flow of air into pneumatic line 110.

In various embodiments, after solenoid B is de-energized, the previously described steps 210 through 220 repeated, but on the upper chambers instead of the lower chambers, in order to retract the pneumatic cylinders. A short description of the steps to retract the pneumatic cylinders, according to various embodiments, will be provided. First, in order to retract the pneumatic cylinders, solenoid A (reference number 140) is energized (step 210), thereby permitting flow through pneumatic line 118 to the upper chambers 78 and 78′. As a result of the flow of air into the upper chambers 78 and 78′, the pneumatic cylinders retract as designated at step 212. The pneumatic cylinders then retract to the intermediate position shown in FIG. 4, where the heated lid is in its fully closed position, but the handle remains in its upward position as shown in FIG. 4. The pneumatic cylinder continues to retract, pivoting the handle 50 from its upward position. In various embodiments, the downward movement can cause the locking device to become engaged. The handle continues to pivot downward until it is in its fully downward position as shown in FIGS. 1-3, the heated lid remaining in its fully closed position. As the handle approaches the fully downward position and the pneumatic cylinders continue to retract, the reed switch positioned toward the bottom of the pneumatic cylinder is activated (step 214 in FIG. 7). As discussed earlier, there can be a predetermined delay (step 216 in FIG. 7) after the reed switch is activated. After the delay, solenoid A is de-energized (step 218 in FIG. 7). There can then be another predetermined delay (step 220 in FIG. 7). The handle is now in its fully downward position and the heated lid is now in its fully closed position shown in FIGS. 1-3. In the position for the handle and heated lid shown in FIGS. 1-3, the thermal cycling device can be operated to perform thermal cycling operations such as polymerase chain reaction (“PCR”) on the biological samples of the sample well tray.

After the thermal cycling operation is completed on the sample well tray, the cycle can be repeated, as described above, in order to repeatedly open the heated lid, remove the sample well tray, insert a new sample well tray, close the heated lid, and perform a new thermal cycling operation. This sequence can be controlled manually or by a controller. In various embodiments, upon an appropriate determination to stop the opening and closing of the heated lid and thermal cycling operations (step 222), a signal can be sent to the system to stop the opening and closing of the heated lid (step 224). The process can be resumed again as shown at step 200.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a charged species” includes two or more different charged species. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the structure and methods described above. Thus, it should be understood that the present teachings are not limited to the examples discussed in the specification. Rather, the present teachings are intended to cover modifications and variations.

1. A system for thermal cycling samples, the system comprising:
at least one thermal cycling device having a plurality of cavities adapted to receive at least a portion of a plurality of sample wells;
at least one heated lid;
at least one pneumatic driver connected to the heated lid, the pneumatic driver configured to position the heated lid in a closed position and an open position, and to move the heated lid between the closed position and the open position;
at least one pneumatic actuator connected to the pneumatic driver, the pneumatic actuator configured to actuate the pneumatic driver to automatically position and move the heated lid between the closed position and the open position; and
at least one controller coupled to the pneumatic actuator, the controller configured to provide at least one of an electric signal and pneumatic signal to the pneumatic actuator to actuate the pneumatic driver.

2. The system of claim 1, comprising a plurality of thermal cycling devices.

3. The system of claim 2, comprising a plurality of heated lids, pneumatic drivers, and pneumatic actuators, each heated lid connected to a corresponding pneumatic driver and pneumatic actuator.

4. The system of claim 3, comprising a plurality of controllers, wherein the plurality of pneumatic actuators are each controlled by a corresponding controller.

5. The system of claim 3, wherein the plurality of pneumatic actuators are controlled by a single controller.

6. The system of claim 5, wherein said single controller comprises a control computer.

7. The system of claim 1, comprising a plurality of controllers.

8. The system of claim 1, wherein each said pneumatic driver comprises at least one reciprocating pneumatic cylinder.

9. The system of claim 1, wherein each said pneumatic driver comprises a pair of reciprocating pneumatic cylinders.

10. The system of claim 8, wherein each pneumatic actuator comprises a flow controller and a multi-position solenoid valve.

11. The system of claim 10, wherein the controller coupled to the pneumatic actuator provides at least one of the electric signal and pneumatic signal to the pneumatic actuator to selectively energize and de-energize the solenoid valve to retract and extend the at least one reciprocating pneumatic cylinder.

12. The system of claim 10, wherein the flow controller of the pneumatic actuator controls the amount of air flow to the pneumatic cylinder.

13. The system of claim 11, wherein the controller coupled to the pneumatic actuator comprises a control circuit.

14. The system of claim 13, wherein the control circuit is part of a control computer.

15. The system of claim 1, wherein the heated lid permits loading of the sample well tray into the thermal cycling device when the heated lid is in the open position.

16. The system of claim 1, wherein the thermal cycling device and heated lid are configured to permit robotic loading and removal of the sample well tray in the thermal cycling device.

17. The system of claim 1, wherein the thermal cycling device and heated lid are configured to permit manual loading and removal of the sample well tray in the thermal cycling device.

18. The system of claim 1, wherein the heated lid is configured to pivot about the thermal cycling device when moved between the closed position and the open position.

19. The system of claim 1, further comprising an air source for the pneumatic driver.

20. The system of claim 19, wherein the pneumatic driver comprises at least one pneumatic cylinder with an upper and lower chamber, the pneumatic actuator configured to permit pre-charging of an empty chamber of the pneumatic cylinder with air prior to a retraction or extension of the pneumatic cylinder.

21. The system of claim 1, wherein the heated lid further comprises a handle pivotably connected to the heated lid, said pneumatic driver being pivotably connected to the handle of the heated lid.

22. The system of claim 21, wherein the pneumatic driver is adapted to both rotate the handle and open the heated lid.

23. The system of claim 1, further comprising a locking device configured to retain the heated lid in the closed position during thermal cycling.

24. The system of claim 23, wherein the pneumatic drive is adapted to both activate the locking device and open the heated lid.

25. The system of claim 24, wherein the locking device comprises a cam mechanism.

26. The system of claim 23, wherein the locking device is electrically actuated to selectively lock the heated lid in a fully closed position.

27. The system of claim 23, wherein the locking mechanism is pneumatically actuated to selectively lock the heated lid in a fully closed position.

28. A method for thermal cycling samples, the method comprising:
providing biological samples in a plurality of sample wells;
positioning the sample wells in a thermal cycling device;
pre-charging a pneumatic driver;
closing a heated lid with the pneumatic driver;
locking the heated lid with the pneumatic driver;
thermally cycling the biological samples;
unlocking the heated lid with the pneumatic driver;
opening the heated lid with the pneumatic driver; and
removing the sample wells from the thermal cycling device.

29. The method of claim 28, wherein closing and locking comprise actuating the pneumatic driver.

30. The method of claim 28, wherein opening and unlocking comprise actuating the pneumatic driver.

31. The method of claim 28, wherein positioning and removing the sample wells comprises robotic manipulation.

32. A system for thermal cycling biological samples, comprising:
a plurality of sample well trays, each sample well tray having a plurality of sample wells;
a plurality of thermal cycling devices, each thermal cycling device having a plurality of cavities to receive at least a portion of the sample wells;
a heated lid for each of the thermal cycling devices;
a pair of pneumatic cylinders connected to each of the heated lids, the pair of pneumatic cylinders configured to position the heated lid in a closed position and an open position, and to move the heated lid between the closed position and the open position;
a pneumatic actuator connected to each pair of pneumatic cylinders, the pair of pneumatic actuators configured to actuate the pneumatic cylinders to position and move the heated lid between the closed position and the open position; and
at least one controller coupled to the pneumatic actuators, the controller comprising a control circuit configured to provide an electric signal to the pneumatic actuator.
